(19) United States
(12) Patent Application Publication
(10) Pub. No.: US 2025/0034498 A1
(43) Pub. Date: Jan. 30, 2025

(54) MICROFLUIDIC DEVICES

(71) Applicant: Ecole Polytechnique Federale de Lausanne, Lausanne (CH)

(72) Inventors: Jisoo PARK, Lausanne (CH); Matthias LUETOLF, St-Sulpice (VD) (CH)

(73) Assignee: Ecole Polytechnique Federale de Lausanne, Lausanne (CH)

(21) Appl. No.: 18/709,474

(22) PCT Filed: Nov. 11, 2021
(86) PCT No.: PCT/EP2021/081433

§ 371 Date: May 10, 2024

(51) Int. Cl. C12M 3/06; C12M 1/00; C12M 1/12; C12N 5/00

(52) U.S. Cl. CPC C12M 23/16; C12M 23/40; C12M 25/14; C12N 5/0062

(57) ABSTRACT

The present disclosure relates to a microfluidic device comprising: a substrate; a culture chamber (130); a loading channel (170) in fluid communication with the culture chamber; at least one auxiliar channel (170-1, 5 170-2) extending from and in fluid communication with the loading channel, wherein the at least one auxiliary channel is so dimensioned such that a hydraulic resistance in the at least one auxiliary channel is higher than a hydraulic resistance in the loading channel; a test area (150) defined along the loading channel at a position between the loading channel and the at least one auxiliary channel; a first medium reservoir (110) in fluid communication with a first side of the test area; and a second medium reservoir (120) in fluid communication with a second side of the test area, the second side being different from the first side.

### FIELD OF THE INVENTION

The present disclosure relates generally to organ/organoid-on-a-chip technology and more specifically to microfluidic devices and methods for patterning a biological sample such as an organoid or a tissue sample.

### BACKGROUND

Organoids and organ/organoid-on-a-chip has become an invaluable tool for drug discovery and drug screening in the pharmaceuticals industry and may play a significant role in the future of personalised medicine applications. To date, precisely delivering molecules (e.g. drugs, morphogens, chemicals, etc.) to three-dimensional (3D) in vitro tissues within microfluidics systems in a spatiotemporally controllable way remains one of the largest challenge of this emerging field.

Conventional technologies focus on the toxicology aspect of organ/organoid-on-a-chip in which molecules under testing are placed on a cell or tissue sample, and their responses are simply screened and observed. However, such conventional organ/organoid-on-a-chip technique does not allow organoids to be loaded and placed at a desired position to be exposed to biochemical gradient and as such cannot achieve spatial patterning of organoids.

In view of the foregoing, it is desirable to provide improved microfluidic devices and methods of using the microfluidic devices that enable patterning of biological samples such as organoids and tissues.

### BRIEF DESCRIPTION OF THE DRAWINGS

Embodiments will now be described, with reference to the accompanying drawings, in which:

FIG. 1 shows an exemplary microfluidic device for organoid patterning according to an embodiment;

FIG. 2 shows examples of gradient window designs;

FIG. 3 shows an exemplary gel/organoid loading channel of the microfluidic device of FIG. 1;

FIG. 4 shows gradient formation by fluorophore at the gradient window of the microfluidic device of FIG. 1;

FIG. 5 shows an example of spatial patterning in an organoid;

FIG. 6 shows an example of organoids responding to morphogen gradients; and

FIG. 7 shows another microfluidic device for organoid patterning according to an alternative embodiment.

### DETAILED DESCRIPTION

An aspect of the present technology provides a microfluidic device comprising: a substrate; a culture chamber; a loading channel in fluid communication with the culture chamber; at least one auxiliar channel extending from and in fluid communication with the loading channel, wherein the at least one auxiliary channel is so dimensioned such that a hydraulic resistance in the at least one auxiliary channel is higher than a hydraulic resistance in the loading channel; a test area defined along the loading channel at a position between the loading channel and the at least one auxiliary channel; a first medium reservoir in fluid communication with a first side of the test area; and a second medium reservoir in fluid communication with a second side of the test area, the second side being different from the first side.

According to embodiments of the present technology, the provision of the at least one auxiliary channel to the microfluidic device increases the overall hydraulic resistance experienced by a fluid as it flows along the loading channel, thus slowing the fluid flow. As such, when a biological sample such as an organoid or a tissue sample is introduced into the device, the sample approaches the test area, or gradient window, at a slower rate compared to conventional technology in which no auxiliary channels are provided. It is therefore possible to position the sample easily and precisely at the test area. As a result of the improved precision with the positioning of the sample, embodiments of the present technology do not require additional size-specific structure to “trap” the sample. Consequently, it is possible to reduce the dimension of the test area and achieve a window size capable of generating a stable gradient of molecule(s) of interest to which the sample is subjected for spatial patterning without e.g. cumbersome flow generating systems. Due to its simplicity, the present technology may be applied to automated systems for large-scale drug screening or organoid development in the pharmaceutical industry and research institutes. Furthermore, improvements to the size and structure of the test area that enables the development of a sharp gradient of the molecule of interest across the test area allows a biological sample such as an organoid to be patterned in a symmetry-breaking way. The present approach enables a tight control over symmetry breaking events that orchestrate organoid patterning. The present technology therefore significantly reduces the gap to bring more accurate in vitro models used in routine for pre-clinical and in the future clinical phases.

In some embodiments, when a medium is introduced into the first or the second medium reservoir, the medium may reach the test area (gradient window) unaided, for example if the device is formed of a hydrophilic material. In other embodiments, the medium may be driven into the test area by one or more different mechanisms. In some embodiments, the device may further comprise: a first air channel in fluid communication with the first medium reservoir arranged to allow air between the first medium reservoir and the test area to depart via a first air outlet; and a second air channel in fluid communication with the second medium reservoir arranged to allow air between the second medium reservoir and the test area to depart via a second air outlet. Evacuating an air gap between the first (second) medium reservoirs and the test area allows a medium inside the first (second) medium reservoir to move in to fill the void left by the air gap, and thus drives the medium into the test area.

While the dimensions of the loading channel are restricted by the size of biological sample, there may be various different ways of determining suitable dimensions for the auxiliary channel to reach an overall hydraulic resistance that provides the desired slowing effect of the sample in the loading channel. In some embodiments, the at least one auxiliary channel may be so dimensioned such that the hydraulic resistance therein equals to or is above a predetermined hydraulic resistance threshold, and/or the at least one auxiliary channel may be so dimensioned such that a flow rate of a fluid in the loading channel is below a predetermined flow rate threshold caused by an increase in hydraulic resistance from the loading channel to the at least one auxiliary channel.

The hydraulic resistance experienced by a fluid in a channel depends, amongst other things, the cross-sectional area and the length of the channel. Thus, in some embodiments, so as to reach a desired hydraulic resistance, the at least one auxiliary channel may have a cross-sectional area smaller than a cross-sectional area of the loading channel, and/or the at least one auxiliary channel may be longer than the loading channel.

In some embodiments, the at least one auxiliary channel may comprise a first auxiliary channel and a second auxiliary channel, wherein the first auxiliary channel and the second auxiliary channel both extend from and in fluid communication with the loading channel.

In some embodiments, the first auxiliary channel and the second auxiliary channel may each have a cross-sectional area smaller than the cross-sectional area of the loading channel, and/or a combined cross-sectional area of the first auxiliary channel and the second auxiliary channel may be the same as or smaller than the cross-sectional area of the loading channel.

In some embodiments, the first auxiliary channel may be arranged to extend into the first medium reservoir and the second auxiliary channel is arranged to extend into the second medium reservoir.

Since it is possible, according to embodiments of the present technology, to position a biological sample in the test area at a slow rate, it is possible for the test area to be arrange in different dimensions, shapes and orientations without significant concerns over the precision of the positioning of the sample. Thus, in some embodiments, the loading channel may be defined by a channel axis along the length of the loading channel, the test area may be defined by a length and a width and comprises a longitudinal axis defined along the length of the test area, and wherein the longitudinal axis of the test area may coincide with the channel axis of the loading channel.

In some embodiments, the loading channel may be defined by a channel axis along the length of the loading channel, the test area may be defined by a length and a width and comprises a longitudinal axis defined along the length of the test area, and wherein the longitudinal axis of the test area may define an angle with the channel axis of the loading channel.

In some embodiments, the device may further comprise a fluid introduction channel in fluid communication with the loading channel for introducing a fluid into the loading channel. Thus, when introducing a biological sample into the loading channel from the culture chamber, it is possible to introduce a fluid such as a hydrogel into the loading channel via the fluid introduction channel, either alternatively or in addition to introducing the fluid from the culture chamber together with the sample.

There may be occasions on which a biological sample is introduced into the loading channel with a medium, whether it is the same medium as the medium to be introduced into the first and/or second medium reservoirs or a different medium. In this case, it may be desirable to create a barrier between the first and second medium reservoirs and the test area such that media within the first and second reservoir may be introduced into the test area with control. In some embodiments, the device may further comprise at least one fluid barrier channel in fluid communication with the test area for forming a fluid barrier in the test area.

In some embodiments, the at least one fluid barrier channel may comprise a first fluid barrier channel for forming a fluid barrier between the first medium reservoir and the test area and a second fluid barrier channel for forming a fluid barrier between the second medium reservoir and the test area.

In some embodiments, the test area may be defined by a length and a width, and the length of the test area may be in a range of 50 μm-5 mm, optionally the length of the test area may be 300 μm.

In some embodiments, the loading channel may be defined by a width, and the width of the loading channel may be in a range of 50 μm-5 mm, optionally the width of the loading channel may be 200 μm.

In some embodiments, the at least one auxiliary channel may be defined by a width, and the width of the at least one auxiliary channel may be in a range of 25 μm-2.5 mm, optionally the width of the at least one auxiliary channel may be 100 μm.

Another aspect of the present technology provides a method of preparing a biological sample for patterning using the microfluidic device described above, the method comprising: (a) introducing the biological sample into the culture chamber; (b) positioning the biological sample at the test area by releasing the biological sample into the loading channel, wherein the biological sample is urged towards the test area by a flow of a bio-compatible gel; and (d) maintaining the device under conditions in which the bio-compatible gel polymerises.

In some embodiments, the biological sample and the bio-compatible gel may be introduced into the culture chamber together; and/or the device comprises a gel introduction channel in fluid communication with the loading channel, the method further comprising introducing the bio-compatible gel into the loading channel via the gel introduction channel.

A further aspect of the present technology provides a method of preparing a biological sample for patterning using the microfluidic device described above, wherein the device comprises a fluid barrier channel in fluid communication with the test area, the method comprising: (a′) introducing the biological sample suspended in a medium into the culture chamber; (b′) positioning the biological sample at the test area by releasing the biological sample into the loading channel, wherein the biological sample is urged towards the test area by a flow of the medium; (c′) introducing a bio-compatible gel via the fluid barrier channel to form a gel barrier around a portion of the test area; and (d′) maintaining the device under conditions in which the bio-compatible gel polymerises.

In some embodiments, the method may further comprise, prior to step (d) or (d′), placing a drop of a medium in the first medium reservoir and/or the second medium reservoir for humidification.

A yet further aspect of the present technology provides a method of patterning a biological sample using the microfluidic device described above, the method comprising: preparing the biological sample according the method described above; (e) filling the first medium reservoir with a first medium supplemented with a first morphogen; (f) opening the first air outlet to remove air from the first medium reservoir via the first air channel to release the first medium into the test area; and (g) incubating the device under conditions in which the biological sample proliferate in the test area in response to a gradient of the first morphogen across the test area.

In some embodiments, the method may further comprise, prior to step (g): filling the second medium reservoir with a second medium supplemented by a second morphogen; and opening the second air outlet to remove air from the second medium reservoir via the second air channel to release the second medium into the test area, such that when the device is being incubated the biological sample proliferates in the test area in response to both the gradient of the first morphogen and a gradient of the second morphogen across the test area.

A yet further aspect of the present technology provides a patterned organoid obtained by the method as described above. The organoid may be any organoids such as (but not limited to) spinal cord organoids, brain organoids, embryoids, etc., and in an example, it may be a neural tube organoid. The present technology is not limited to organoids but may include other human and animal tissues.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### First Embodiment

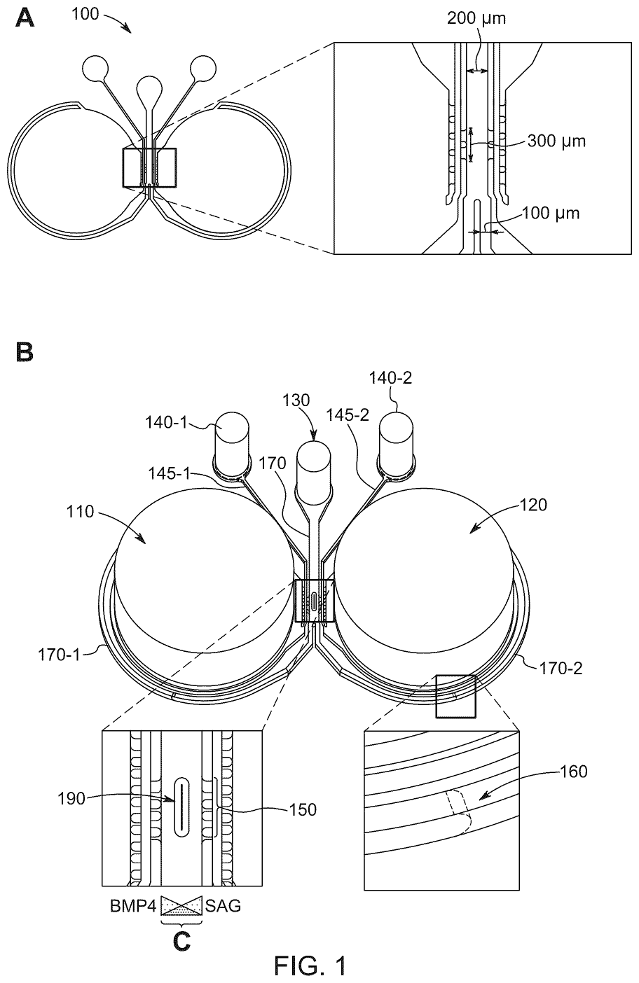

FIG. 1 shows an exemplary microfluidic device 100 for organoid patterning according to an embodiment of the present technology. Elements of the microfluid device 100 may be provided on a substrate. The microfluidic device 100 comprises a first medium chamber or reservoir 110 containing a first medium, which may for example be supplemented with a first morphogen or signalling molecule, and a second medium chamber or reservoir 120 containing a second medium, which may for example be supplemented with a second morphogen or signalling molecule. For example, BMP4 as a dorsalizing morphogen in chamber 110 and SHH as a ventralizing morphogen in chamber 120 can be applied to recapitulate the dorsal-ventral patterning of neural tube in vivo. For example, the first morphogen or signalling molecule may be a dorsalizing morphogen such as Bone Morphogenetic Protein 4 (BMP4), and the second morphogen or signalling molecule may be a ventralizing morphogen such as SHH or SAG. While it is preferable for the first and second morphogen or signalling molecule to be different, it is not essential, and in some embodiments, they may be the same morphogen or signalling molecule, for example the same morphogen or signalling molecule may be provided at different concentrations. The microfluidic device 100 further comprises a culture chamber 130 for loading an organoid or tissue. The culture chamber 130 is in fluid communication with a loading channel 170 that leads to a gradient window 150, then the loading channel 170 splits into two narrower channels, a first auxiliary channel 170-1 and a second auxiliary channel 170-2. Herein, a gradient window refers to a test area at a position along the loading channel 170 where an organoid or tissue is to be placed so as to be subjected to a concentration gradient of a test substance. The first and second chambers 110, 120 are arranged to respectively supply the first medium and the second medium into the gradient window 150. The first and second media diffuse across the gradient window 150 in opposite directions to create a gradient of the respective medium across the gradient window 150 indicated by C in FIG. 1.

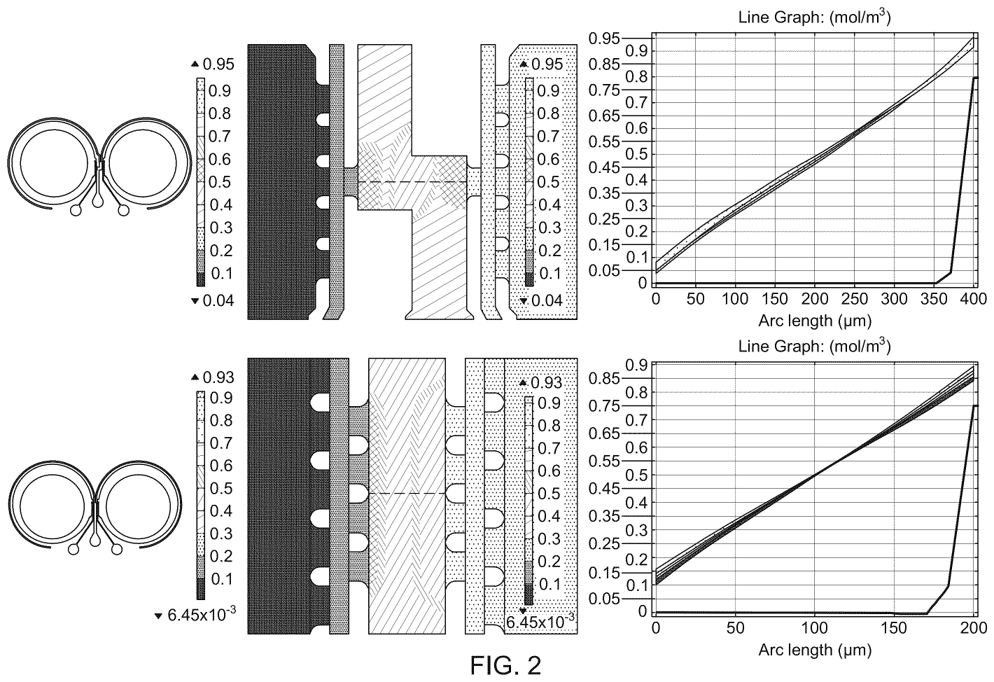

To load an organoid or tissue, e.g. organoid 190, into the gradient window 150, the organoid suspended in a fluid substrate (e.g. a bio-compatible gel such as a hydrogel or Matrigel™, a medium, etc.) is placed in the culture chamber 130. In some embodiments, an organoid or tissue can be placed in the culture chamber 130 without the fluid substrate for example by providing an additional chamber (not shown) adjacent the culture chamber 130 for introducing the fluid substrate into the culture chamber 130 or the loading channel 170 via a fluid substrate channel (not shown) that leads from the additional chamber to the culture chamber 130 or the loading channel 170 to supply the organoid with the fluid substrate. The organoid 190 is carried towards the gradient window 150 by the flow of the fluid substrate along the loading channel 170 towards the first and second auxiliary channels 170-1, 170-2. The auxiliary channels 170-1 and 170-2 function as an extension of the organoid loading channel 170 to allow the fluid substrate a continuous passage. Moreover, the first and second auxiliary channels are dimensioned such that the hydraulic resistance in the auxiliary channels is higher than the hydraulic resistance in the organoid loading channel 170 (this will be explained below with reference to FIG. 2). The organoid loading channel 170 and the auxiliary channels 170-1, 170-2 of the present embodiments therefore act in combination to slow down the progression of the organoid 190 towards the gradient window 150 through an increase in hydraulic resistance, and furthermore allow the fluid substrate to continue to flow until the organoid 190 is in place, thus giving a user more control and more time to precisely position the organoid in the gradient window 150. When in position, the organoid 190 is stopped at the gradient window 150 by ceasing the flow of the fluid substrate. It should be noted that, while the present embodiment depicts two auxiliary channels 170-1, 170-2, similar technical effects may be achieved by embodiments in which only a single auxiliary channel or more than two auxiliary channels are provided.

According to the embodiments, the channels 170, 170-1 and 170-2 are air-filled before loading. Thus, as the fluid substrate fills the organoid loading channel 170 and optionally the auxiliary channels 170-1, 170-2, there exists an air gap between the gradient widow 150 and each of the first medium chamber 110 and the second medium chamber 120, preventing the first and second media from reaching the gradient window 150. To this end, the microfluidic device 100 further comprises first and second air outlets 140-1 and 140-2 in respective fluid communication with a first air channel 145-1 and a second air channel 145-2 for removing such air gaps within the device 100 so as to promote flow of the first and second media into the gradient window 150.

In use, an organoid is placed together with a fluid substrate such as a hydrogel in the culture chamber 130 and allowed to flow into the organoid loading channel 170. Loading is ceased when the organoid is precisely positioned in the gradient window 150. The first and second air outlets 140-1, 140-2 are then opened to remove air gaps between the first and second chambers 110, 120 and the gradient window 150 via the first and second air channel 145-1, 145-2 to allow the first and second media to reach the gradient window 150. The first and second media are allowed to diffuse across the gradient window 150 and a concentration gradient of the morphogen (or signalling molecule) in the respective media is developed. The hydrogel is then allowed to polymerise or solidify before observation.

The shapes, proportions and dimensions of various elements of the microfluidic device 100 shown in FIG. 1 are intended as examples only and should not be considered essential in any way. In some embodiments, the range of dimension for various elements of the device may be determined by the organoid of interest. For example, for intestinal organoids, the size range may be around 150-400 μm, while for brain organoids, the size range may be much bigger at about 1 mm to 5 mm. The various channels may have a circular, rectangular or any other shape cross-section, and the width or diameter of the various channels may vary depending on the type of organoid or tissue in question. For example, the width or diameter of the various channels may range from 50 μm to 5 mm. The shape and size of the gradient window 150 for tissue patterning is not limited to the shape and size shown in the embodiment of FIG. 1; other shapes and sizes are possible if desired for various other applications. Some examples of gradient window shapes and sizes are shown in FIG. 2. The design for the gradient window is not limited to the shapes and sizes shown herein due to the ability of the present technique to precisely position an organoid. For reference, gradient formation simulations by COMSOL Multiphysics for the two gradient window designs are shown.

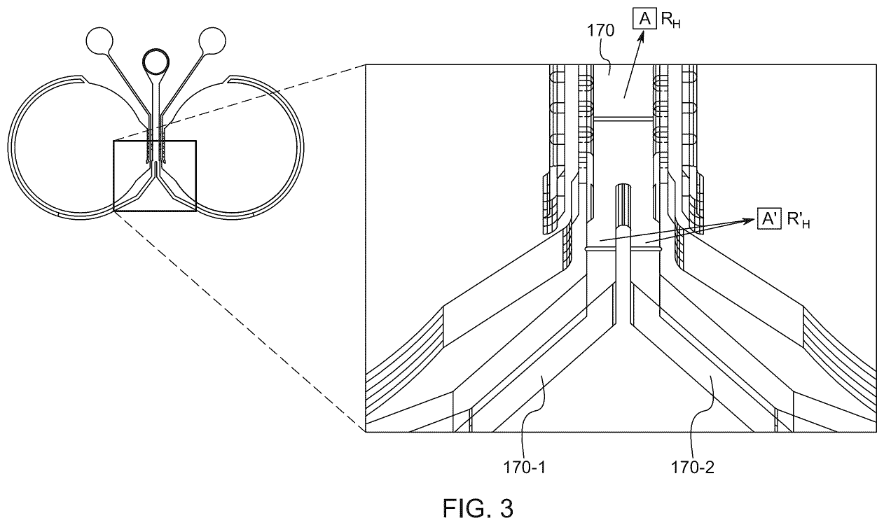

FIG. 3 illustrates the organoid loading channel 170 and the first and second auxiliary channels 170-1, 170-2 in more detail. As described with reference to FIG. 1, the organoid loading channel 170 leads from the culture chamber 130 towards the gradient window 150 and is then divided into two auxiliary channels 170-1, 170-2. The hydraulic resistance R_{H} in a channel is determined by the equation:

$${R}_{H} = β \frac{η L}{{A}^{2}} \qquad [ Equation 1 ]$$

where β is a geometric correction factor, η denotes a fluid viscosity, L denotes the length of the channel, and A denotes the cross-sectional area of the channel. In the present embodiment, the cross-sectional area A of the loading channel 170 is larger than the cross-sectional area A′ of the two auxiliary channels 170-1, 170-2. Since the hydraulic resistance in a channel is inversely proportional to the square of the cross-sectional area of the channel, it can be seen that the hydraulic resistance in the two auxiliary channels 170-1, 170-2 is higher than the hydraulic resistance in the loading channel 170, R_{H}.

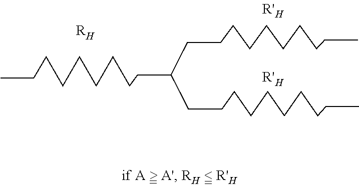

As a consequence of the increase in hydraulic resistance following the division from the loading channel 170 to two auxiliary channels 170-1 and 170-2, the flow rate of the fluid substrate in the loading channel 170 is reduced compared to cases where the hydraulic resistance before and after the gradient window 150 remains the same throughout. Thus, according to the present embodiment, the organoid 190 is restricted to travel along the loading channel 170 slowly as it approaches the gradient window 150 as the fluid substrate in which the organoid 190 is suspended flows along the loading channel 170 and optionally into the auxiliary channels 170-1, 170-2 as a result of the difference in hydraulic resistance, and the slow approach enables the organoid 190 to be precisely placed at the desired position in the gradient window 150. By configuring and dimensioning the loading channel 170 and auxiliary channels 170-1 170-2 to achieve a higher hydraulic resistance within the channel(s), it is possible for embodiments of the present technology to enable the positioning of an organoid at the desired loading position with improved precision. It should be noted that the position at which the loading channel 170 becomes one or more auxiliary channels is not essential for achieving the technical effects of the present technique, as long as any narrowing of the loading channel 170 is beyond the desired loading position (e.g. the gradient window) of an organoid to allow a free passage for the organoid to reach the loading position.

### EXPERIMENTAL EXAMPLES

The following describes an implementation example of dorsal-ventral patterning of neural tube organoids using a microfluidic device such as the device 100 according to embodiments of the present technology.

The microfluidic device may be fabricated using standard photolithography and soft lithography methods. In order to facilitate visualization, the microfluidic device is typically comprised of a substrate made of an optically transparent material, such as plastic, glass, or polymers, e.g. SU-8. In the present example, the microfluidic device is formed of a first layer of gel loading pillars that is 150 μm in thickness and a second layer configured for phase guidance that is 50 μm thickness using SU-8.

In the present example, the organoid of interest was cultured in a Matrigel™ drop for six days then harvested, and the organoid was suspended in Matrigel™ and loaded into the culture chamber 130 and placed at the gradient window 150 via the loading channel 170. A small drop of N2B27 medium was placed in each of the first and second medium chambers or reservoirs 110, 120 for humidification, and the chip was incubated in a 5% CO₂ and 37° C. incubator for 5 minutes to allow the Matrigel™ to polymerise. After incubation, the first and second chambers 110, 120 were filled with N2B27 medium supplemented with 1.6 nM of BMP4 as a dorsal morphogen and 500 nM of SAG as a ventral morphogen respectively, and residual air in the device was removed from the first and second air outlets 140-1, 140-2 to allow the morphogen-supplemented media to diffuse across the gradient window 150. The device containing the organoid was incubated in a 5% CO₂ and 37° C. incubator for 18 hours.

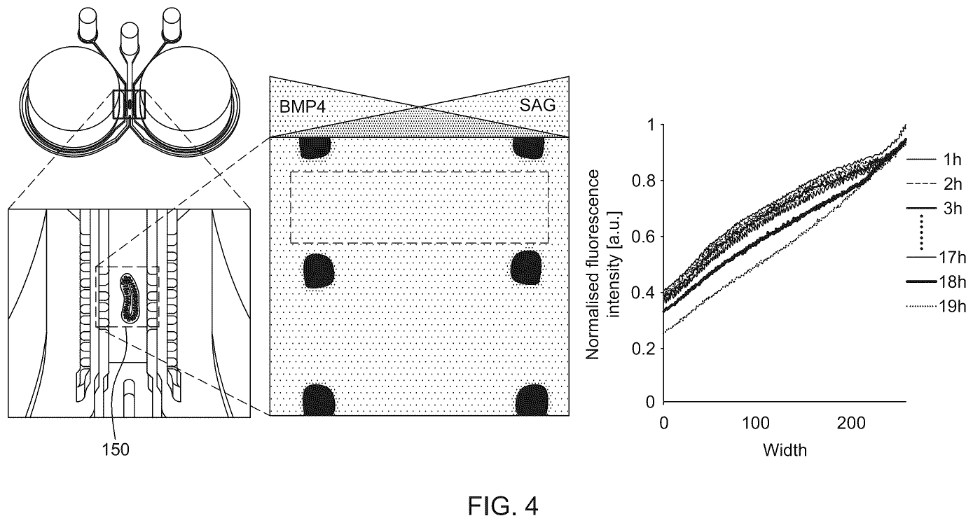

FIG. 4 shows the experimental result of gradient formation at the gradient window in the microfluidic chip. Gradient formation at the gradient window was experimentally validated by time-lapse imaging of fluorophore diffusion over 19 hours on a confocal microscope and the fluorescence intensity was quantified every hour. The present example shows the result of 20 kDa fluorophore gradient. However, it will be clear to a skilled person that this can be done with other size of fluorophore to model the diffusion of the morphogen of interest according to its molecular weight.

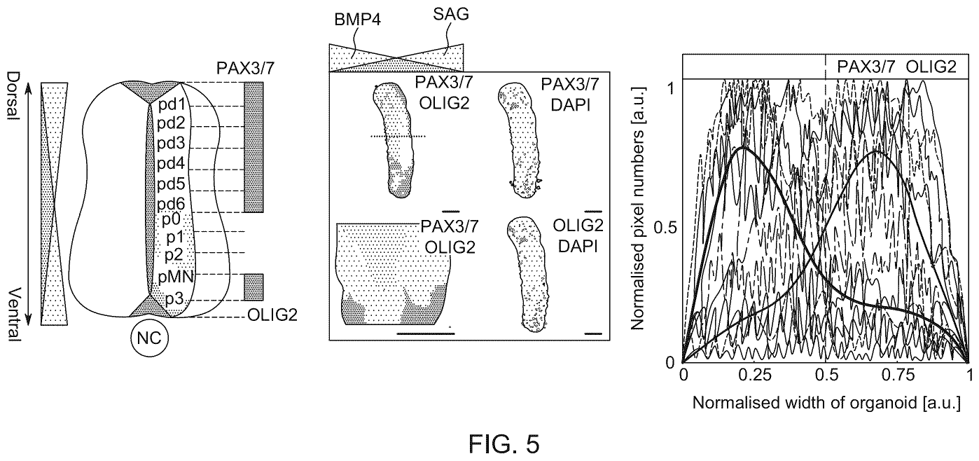

FIG. 5 shows a dorsal-ventral patterned neural tube organoid as an example of spatial patterning in organoid based on the experiment described above. A neural tube organoid was placed in the gradient window of the device where a dorsal-ventral morphogen gradient has developed, using BMP4 as a dorsal morphogen and SAG as a ventral morphogen. PAX3/7 was used as a marker for dorsal fate neural tube cell and OLIG2 was used as a marker for ventral fate neural tube cell. As can be seen in FIG. 5, the organoid exposed to this gradient showed symmetry-breaking spatial patterning, where PAX3/7 positive cells were biased towards the BMP4 source and OLIG2 positive cells were biased towards the SAG source. This demonstrates the potential for application of the present technology for spatial patterning in organoids and drug screening.

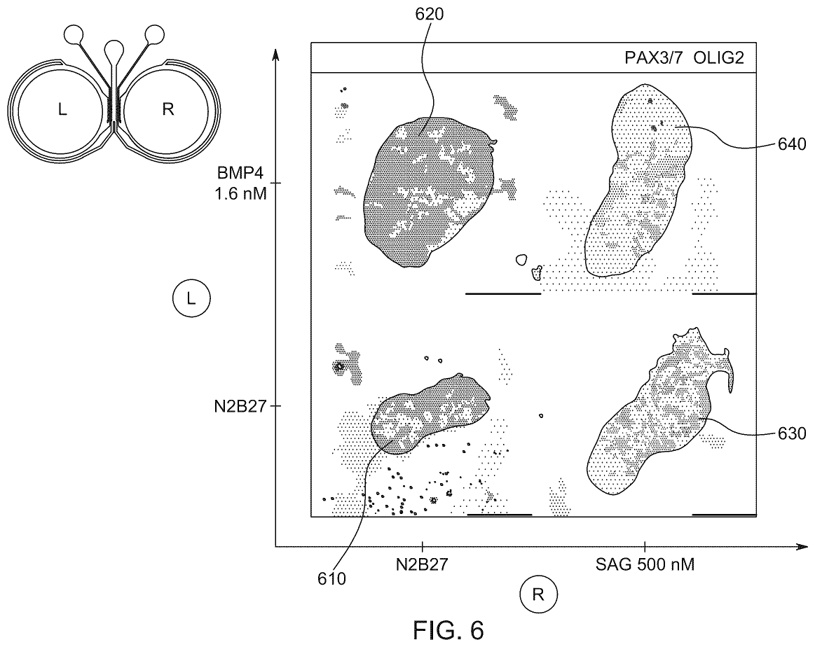

FIG. 6 shows the response of neural tube organoids to morphogen gradient. As described above, the neural tube organoids from D6 were introduced in the microfluidic device and exposed to different morphogen gradients for 18 hours. All scale bars are 100 μm. The control organoid 610 with no morphogen exposure (N2B27 (Left; L)-N2B27 (Right; R)) shows a default dorsal identity expressing only PAX3/7+ cells. The organoid 620 exposed only to dorsal morphogen BMP4 (1.6 nM BMP4 (L)-N2B27 (R)) also shows the dorsal identity expressing PAX3/7. Meanwhile, the organoid 630 exposed to ventral morphogen SAG (N2B27 (L)-500 nM SAG (R)) shows dominant ventral identity expressing OLIG2. Finally, the organoid 640 exposed to the anti-parallel gradient of BMP4 and SAG (1.6 nM BMP4 (L)-500 nM SAG (R)) shows symmetry-breaking patterning along the gradient axis expressing PAX3/7 biased to the left side and OLIG2 biased to right side.

The above examples demonstrate that a morphogen gradient created at a gradient window of a microfluidic device according to embodiments of the present technology enables spatial patterning in a single organoid.

### Second Embodiment

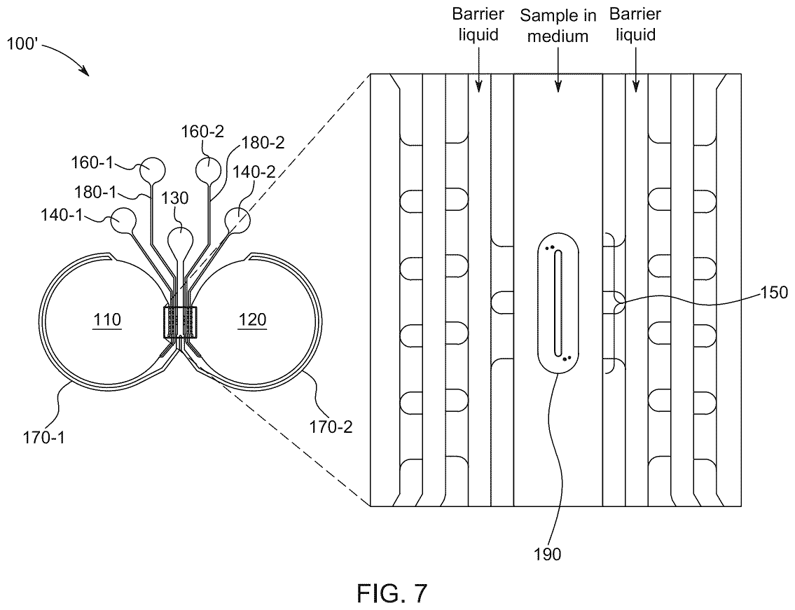

In an alternative embodiment, as shown in FIG. 7, additional channels—a first fluid barrier channel 180-1 and a second fluid barrier channel 180-2—are provided, arranged to direct a fluid from respective first barrier fluid inlet 160-1 and second barrier fluid inlet 160-2 to the space between the gradient window 150 and each of the first medium chamber or reservoir 110 and the second medium chamber or reservoir 120 respectively.

In the present embodiment, an organoid may be suspended in a medium instead of a hydrogel (or other bio-compatible gel) when placed in the culture chamber 130. In particular, the medium in which the organoid is suspended may be the same medium as the first and second media used in the first and second medium chambers 110, 120 but without a morphogen/signalling molecule supplement. In this case, in order to allow a morphogen gradient to develop across the gradient window 150, a barrier of viscous liquid capable of solidifying or polymerising to allow a slow diffusion of morphogens or signalling molecules, such as a hydrogel, is placed between the organoid/medium and the first and second medium reservoirs. To enable the placement of the fluid barrier, the first and second barrier channels 180-1, 180-2 are arranged to insert a small amount (e.g. a few μL) of barrier fluid between the first medium in the first chamber 110 and the gradient window 150 and between the second medium in the second chamber 120 and the gradient window 150.

In use, after an organoid suspended in a medium is positioned in the gradient window 150, a barrier fluid is introduced between the first medium reservoir 110 via the first fluid barrier channel 180-1 at the first barrier fluid inlet 160-1, and between the second medium reservoir 120 via the second fluid barrier channel 180-2 at the second barrier fluid inlet 160-2. The barrier fluid may for example be a hydrogel such as Matrigel™. The hydrogel is allowed to solidify to create a barrier between the gradient window and the two reservoirs 110, 120. Once the barrier is created, the first and second air outlets 140-1 and 140-2 can be open to release the air trapped in the device to allow the first and second media to diffuse through the hydrogel barrier into the gradient window 150 as described above.

The technology described herein enable precise positioning of an organoid or tissue at a gradient window through the provision of at least one auxiliary channel that enables a continuous flow of a fluid substrate in which the organoid is suspended. Moreover, the at least one auxiliary channel is so dimensioned to increase the hydraulic resistance experienced by the liquid substrate as it flows so that the flow rate of the liquid substrate is reduced, which enables the precision of the positioning of the organoid to be further improved. As such, the technology described herein provides devices and methods to load and place organoids or tissues at a desired position with precision to be exposed to a biochemical gradient, thus enabling spatial patterning of organoids and tissues.

The examples and conditional language recited herein are intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its scope as defined by the appended claims.

Furthermore, as an aid to understanding, the above description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to limit the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the technology, as well as specific examples thereof, are intended to encompass both structural and functional equivalents thereof, whether they are currently known or developed in the future.

It will be clear to one skilled in the art that many improvements and modifications can be made to the foregoing exemplary embodiments without departing from the scope of the present techniques.

1. A microfluidic device comprising:
a substrate;
a culture chamber;
a loading channel in fluid communication with the culture chamber;
at least one auxiliary channel extending from and in fluid communication with the loading channel, wherein the at least one auxiliary channel is so dimensioned such that a hydraulic resistance in the at least one auxiliary channel is higher than a hydraulic resistance in the loading channel;
a test area defined along the loading channel at a position between the loading channel and the at least one auxiliary channel;
a first medium reservoir in fluid communication with a first side of the test area; and
a second medium reservoir in fluid communication with a second side of the test area, the second side being different from the first side.

2. The device of claim 1, further comprising:
a first air channel in fluid communication with the first medium reservoir arranged to allow air between the first medium reservoir and the test area to depart via a first air outlet; and
a second air channel in fluid communication with the second medium reservoir arranged to allow air between the second medium reservoir and the test area to depart via a second air outlet.

3. The device of claim 1, wherein the at least one auxiliary channel is so dimensioned such that the hydraulic resistance therein equals to or is above a predetermined hydraulic resistance threshold, and/or the at least one auxiliary channel is so dimensioned such that a flow rate of a fluid in the loading channel is below a predetermined flow rate threshold caused by an increase in hydraulic resistance from the loading channel to the at least one auxiliary channel.

4. The device of claim 1, wherein the at least one auxiliary channel has a cross-sectional area smaller than a cross-sectional area of the loading channel, and/or the at least one auxiliary channel is longer than the loading channel.

5. The device of claim 1, wherein the at least one auxiliary channel comprises a first auxiliary channel and a second auxiliary channel, wherein the first auxiliary channel and the second auxiliary channel both extend from and in fluid communication with the loading channel.

6. The device of claim 5, wherein the first auxiliary channel and the second auxiliary channel each has a cross-sectional area smaller than a cross-sectional area of the loading channel, and/or wherein a combined cross-sectional area of the first auxiliary channel and the second auxiliary channel is the same as or smaller than the cross-sectional area of the loading channel.

7. The device of claim 5, wherein the first auxiliary channel is arranged to extend into the first medium reservoir and the second auxiliary channel is arranged to extend into the second medium reservoir.

8. The device of claim 1, wherein the loading channel is defined by a channel axis along the length of the loading channel, the test area is defined by a length and a width and comprises a longitudinal axis defined along the length of the test area, and wherein:
the longitudinal axis of the test area coincides with the channel axis of the loading channel, or
the longitudinal axis of the test area forms an angle with the channel axis of the loading channel.

9. The device of claim 1, further comprising a fluid introduction channel in fluid communication with the loading channel for introducing a fluid into the loading channel.

10. The device of claim 1, further comprising at least one fluid barrier channel in fluid communication with the test area for forming a fluid barrier in the test area.

11. The device of claim 10, wherein the at least one fluid barrier channel comprises a first fluid barrier channel for forming a fluid barrier between the first medium reservoir and the test area and a second fluid barrier channel for forming a fluid barrier between the second medium reservoir and the test area.

12. The device of claim 1, wherein the test area is defined by a length and a width, and the length of the test area is in a range of 50 μm-5 mm, optionally the length of the test area is 300 μm.

13. The device of claim 1, wherein the loading channel is defined by a width, and the width of the loading channel is in a range of 50 μm-5 mm, optionally the width of the loading channel is 200 μm.

14. The device of claim 1, wherein the at least one auxiliary channel is defined by a width, and the width of the at least one auxiliary channel is in a range of 25 μm-2.5 mm, optionally the width of the at least one auxiliary channel is 100 μm.

15. A method of preparing a biological sample for patterning using the microfluidic device of claim 1, the method comprising:
(a) introducing the biological sample into the culture chamber;
(b) positioning the biological sample at the test area by releasing the biological sample into the loading channel, wherein the biological sample is urged towards the test area by a flow of a bio-compatible gel; and
(d) maintaining the device under conditions in which the bio-compatible gel polymerises.

16. The method of claim 15, wherein
the biological sample and the bio-compatible gel are introduced into the culture chamber together; and/or
the device comprises a gel introduction channel in fluid communication with the loading channel, the method further comprising introducing the bio-compatible gel into the loading channel via the gel introduction channel.

17. The method of claim 15, further comprising, prior to step (d), placing a drop of a medium in the first medium reservoir and/or the second medium reservoir for humidification.

18. A method of patterning a biological sample using the microfluidic device of claim 2, the method comprising:
preparing the biological sample by:
(a) introducing the biological sample into the culture chamber;
(b) positioning the biological sample at the test area by releasing the biological sample into the loading channel, wherein the biological sample is urged towards the test area by a flow of a bio-compatible gel; and
(d) maintaining the device under conditions in which the bio-compatible gel polymerises;
(e) filling the first medium reservoir with a first medium supplemented with a first morphogen;
(f) opening the first air outlet to remove air from the first medium reservoir via the first air channel to release the first medium into the test area; and
(g) incubating the device under conditions in which the biological sample proliferate in the test area in response to a gradient of the first morphogen across the test area.

19. The method of claim 18, further comprising, prior to step (g):
filling the second medium reservoir with a second medium supplemented by a second morphogen; and
opening the second air outlet to remove air from the second medium reservoir via the second air channel to release the second medium into the test area, such that when the device is being incubated the biological sample proliferates in the test area in response to both the gradient of the first morphogen and a gradient of the second morphogen across the test area.

20. A patterned organoid obtained by the method of claim 18.
